(19) United States
(12) Patent Application Publication
(10) Pub. No.: US 2025/0357002 A1
(43) Pub. Date: Nov. 20, 2025

(54) METHOD AND APPARATUS FOR DETERMINING METASTASIS TISSUE OF CANCER BASED ON LINKED MULTIPLE NEURAL NETWORK MODELS

(71) Applicant: URBAN DATA LAB, Daejeon (KR)

(72) Inventor: Chi Sung AN, Seoul (KR)

(21) Appl. No.: 18/771,691

(22) Filed: Jul. 12, 2024

Related U.S. Application Data
(63) Continuation of application No. PCT/KR2024/007849, filed on Jun. 10, 2024.

(30) Foreign Application Priority Data
May 20, 2024 (KR) 10-2024-0065202
May 20, 2024 (KR) 10-2024-0065203
May 20, 2024 (KR) 10-2024-0065204

(51) Int. Cl. G16H 50/20; G06N 3/0455; G06T 7/00; G06T 7/246; G06V 10/82

(52) U.S. Cl. CPC G16H 50/20; G06N 3/0455; G06T 7/0012; G06T 7/248; G06V 10/82; G06T 2207/20081; G06T 2207/20084; G06T 2207/30096; G06V 2201/032

(57) ABSTRACT

A computer program stored on a computer-readable storage medium may be provided. A method of performing a cancer metastasis tissue determination apparatus operated by a processor may be provided. The method may be comprise obtaining a pathology image including tissue to be determined as cancer; generating a plurality of patches by dividing the pathology image into a preset size; determining a probability that each of the plurality of patches includes tumor tissue by inputting the plurality of patches into a first neural network model trained to distinguish whether the pathology image includes tumor tissue; selecting a patch to be observed from among the plurality of patches based on the probability; and determining whether the pathology image includes tumor tissue or a location of the tumor tissue by inputting the selected patch into a second neural network model trained based on multiple patches to determine whether a specific patch contains tumor tissue.

### CROSS-REFERENCE TO RELATED APPLICATIONS

This application is a Continuation of Application No. PCT/KR2024/007849, filed on Jun. 10, 2024, which in turn claims the benefit of Korean Patent Applications No. 10-2024-0065202, filed on May 20, 2024, No. 10-2024-0065203, filed on May 20, 2024, and No. 10-2024-0065204, filed on May 20, 2024. The entire disclosures of all these applications are hereby incorporated by reference.

### TECHNICAL FIELD

The present invention relates to a technology for determining cancer metastatic tissue by linking two or more neural network models having different characteristics.

### BACKGROUND ART

Cancer diagnosis and treatment are key challenges in the medical field. Although many studies and technologies have contributed to cancer diagnosis and treatment, cancer diagnosis and treatment still remain as one of the major challenges that humanity must overcome.

Existing cancer tissue diagnosis technologies are centered on histopathological examinations, which depend on the subjective judgment and experience of experts.

Recently, cancer diagnosis methods utilizing computer vision and pattern recognition technologies have been proposed due to the development of deep learning and machine learning technologies. The utilization of neural network models due to the development of machine learning technologies presents new possibilities in the fields of medical imaging and diagnosis thereof.

In particular, computer vision and pattern recognition using deep learning technologies are gaining much attention in pathological tissue image analysis. These technologies may be used to extract features from high-resolution tissue images, detect lesions, and classify diseases.

Meanwhile, most studies utilizing neural network models mainly focus on diagnosing cancer tissue using a single neural network, but there are several limitations in diagnosing cancer tissue using a single neural network.

First, due to the complexity and diversity of tissue images, it may be difficult for a single neural network to accurately distinguish or classify all types of cancer tissues. In particular, the shapes and features of various cancer tissues may limit the generalization ability of a single neural network model.

Second, extracting and analyzing various features considering the various characteristics of cancer tissues is a complex task. In order to sufficiently training these complex characteristics using a single neural network, a very large dataset and complex architecture may be required. This may increase the computation and resources required for training and executing the model, which may reduce its practicality.

Finally, an overfitting problem that may occur when diagnosing cancer tissue using a single neural network should also be considered. In particular, when the training dataset is small or imbalanced, the model may become overly dependent on specific features or patterns, which may reduce the generalization ability.

Accordingly, the present invention proposes a technology to overcome the limitations of cancer tissue diagnosis using a single neural network.

### DESCRIPTION OF EMBODIMENTS

### Technical Problem

Provided is a more accurate and reliable diagnostic technology by linking two or more neural networks trained in different ways and using them for cancer diagnosis.

### Solution to Problem

As an embodiment of the present disclosure, a method of performing a cancer metastasis tissue determination apparatus operated by a processor may be provided.

The method according to an embodiment of the present disclosure may comprise obtaining a pathology image including tissue to be determined as cancer; generating a plurality of patches by dividing the pathology image into a preset size; determining a probability that each of the plurality of patches includes tumor tissue by inputting the plurality of patches into a first neural network model trained to distinguish whether the pathology image includes tumor tissue; selecting a patch to be observed from among the plurality of patches based on the probability; and determining whether the pathology image includes tumor tissue or a location of the tumor tissue by inputting the selected patch into a second neural network model trained based on multiple patches to determine whether a specific patch contains tumor tissue.

The first neural network model according to an embodiment of the present disclosure may composed of a neural network with a multiple instance learning (MIL) structure and trained based on training data labeled with a single BAG class that only specifies whether the pathology image includes an instance corresponding to tumor tissue, and output a probability that input data includes the instance.

The selecting a patch to be observed according to an embodiment of the present disclosure may comprise: classifying patches determined to have the probability greater than a certain threshold; and arranging the classified patches in order of high probability.

The second neural network model according to an embodiment of the present disclosure may be composed of a neural network with a recurrent neural network (RNN) structure and trained to determine whether tumor tissue is included in a patch by identifying changes in order of input patches and a spatial relationship between the input patches, and output a probability that the classified patches include tumor tissue when the classified patches are input in order in which they are arranged.

The second neural network model according to an embodiment of the present disclosure may be composed of a neural network with an autoencoder structure including an encoder and a decoder and trained to encode and decode input data based on training data of a pathology image including only normal tissue and restore the input data, and determines a patch in which a restoration error is greater than a preset value when receiving the selected patch and performing encoding and decoding by a location of tumor tissue in the pathology image.

The second neural network model according to an embodiment of the present disclosure may be composed of a neural network with an autoencoder structure including two encoders and one decoder trained based on different training data and trained to encode and decode input data based on training data of a pathology image including only normal tissue and restore the input data, and determines that the pathology image includes tumor tissue if standard deviation for difference values of respective restoration errors by the two encoders is greater than or equal to a preset value when receiving the selected patch and performing encoding and decoding.

The generating a plurality of patches according to an embodiment of the present disclosure may comprise: determining a border of tissue included in the pathology image; removing data of an external area of the border of the tissue; and generating a patch by dividing an internal area of the border of the tissue into a preset size.

The generating a plurality of patches according to an embodiment of the present disclosure, may comprise: after generating the patch, when a tissue area included in the patch is 30% or more and 50% or less of the patch, making the tissue area included in the patch symmetrical left-right or up-down within the patch.

The generating a plurality of patches according to an embodiment of the present disclosure may comprise, after generating the patch, when a tissue area included in the patch is less than 30% of the patch, copying the tissue area included in the patch and pasting the tissue area into a blank area.

A cancer metastasis tissue determination apparatus according to an embodiment of the present disclosure may comprise: a memory including an instruction; and a processor for performing a certain operation based on the instruction, wherein the operation of the processor may comprise: obtaining a pathology image including tissue to be determined as cancer; generating a plurality of patches by dividing the pathology image into a preset size; determining a probability that each of the plurality of patches includes tumor tissue by inputting the plurality of patches into a first neural network model trained to distinguish whether the pathology image includes tumor tissue; selecting a patch to be observed from among the plurality of patches based on the probability; and determining whether the pathology image includes tumor tissue or a location of the tumor tissue by inputting the selected patch into a second neural network model trained to determine whether the plurality of patches include tumor tissue.

### Advantageous Effects of Disclosure

The present invention may more effectively handle the diversity and complexity of cancer tissues by linking two or more neural networks trained in different ways and using them for cancer diagnosis, and may alleviate an overfitting problem of a neural network model and improve the generalization ability.

In addition, because the neural network model of the present invention may be trained in different ways for an identical training data set, the computation and resources required for training and executing the model may be reduced, thereby improving practicality, and because neural network models with different special characteristics are linked, a diagnosis may be made by considering various characteristics of cancer tissues.

Therefore, the present invention may greatly contribute to the development of medical technology by achieving practical application of deep learning and machine learning technology in the field of histopathological examination and at the same time greatly improving the accuracy and efficiency of cancer tissue diagnosis.

### BRIEF DESCRIPTION OF DRAWINGS

FIG. 1 is a configuration diagram of a cancer metastasis tissue determination apparatus, according to an embodiment.

FIG. 2 is a flowchart showing operations performed by a cancer metastasis tissue determination apparatus, according to an embodiment.

FIG. 3 is an exemplary view of an operation of removing data other than tissue by recognizing the border of tissue, according to an embodiment.

FIG. 4 is an exemplary view of an operation of filtering and removing border recognition of an outlier such as a bubble that occurred in tissue in the embodiment of FIG. 3.

FIG. 5 is an exemplary view of an operation of dividing an area corresponding to tissue in a pathology image into a preset size and generating a plurality of patches, according to an embodiment.

FIG. 6 is a view of an embodiment of modifying a patch when a tissue area included in a patch is 30% or more and 50% or less.

FIG. 7 is a view of an embodiment of modifying a patch when a tissue area included in a patch is less than 30%.

FIG. 8 is an exemplary view of a first neural network model that determines the probability that a specific instance is included in a patch, according to an embodiment.

FIG. 9 is an exemplary view of a second neural network model configured in the form of a recurrent neural network (RNN) and operating, according to an embodiment.

FIGS. 10 and 11 are exemplary views of a second neural network model configured in the form of an autoencoder including one encoder and one decoder and operating, according to an embodiment.

FIGS. 12 to 14 are exemplary views of a second neural network model configured in the form of an autoencoder including two encoders and one decoder and operating, according to an embodiment.

### MODE OF DISCLOSURE

Hereinafter, embodiments of the present invention will be described with reference to the drawings. In the following description, descriptions of a well-known technical configuration in relation to a lead implantation system for a deep brain stimulator will be omitted. For example, descriptions of the configuration/structure/method of a device or system commonly used in deep brain stimulation, such as the structure of an implantable pulse generator, a connection structure/method of the implantable pulse generator and a lead, and a process for transmitting and receiving electrical signals measured through the lead with an external device, will be omitted. Even if these descriptions are omitted, one of ordinary skill in the art will be able to easily understand the characteristic configuration of the present invention through the following description.

FIG. 1 is a configuration diagram of a cancer metastasis tissue determination apparatus 100 (Hereinafter referred to as ‘apparatus 100’), according to an embodiment.

Referring to FIG. 1, the apparatus 100 according to an embodiment may include a memory 110, a processor 120, an input/output interface 130, and a communication interface 140.

The memory 110 may store data obtained from an external device or data generated automatically. The memory 110 may store instructions that may perform the operation of the processor 120. For example, the memory 110 may store a pathology image of a specific tissue of a patient, and a first neural network model and a second neural network model to be described later.

The processor 120 is a computing device that controls operations. The processor 120 may execute the instructions stored in the memory 110. The operation of the apparatus 100 according to an embodiment of the present invention can be understood as an operation performed by the processor 120.

The input/output interface 130 may include a hardware interface or software interface that inputs or outputs information.

The communication interface 140 may transmit and receive information through a communication network. To this end, the communication interface 140 may include a wireless communication module or a wired communication module.

The apparatus 100 may be implemented as various types of apparatuses that may perform operations through the processor 120 and transmit and receive information through a network. For example, the apparatus 100 may be implemented in the form of a server, a computer device, a portable communication device, a smart phone, a portable multimedia device, a laptop computer, a tablet PC, etc., but is not limited thereto.

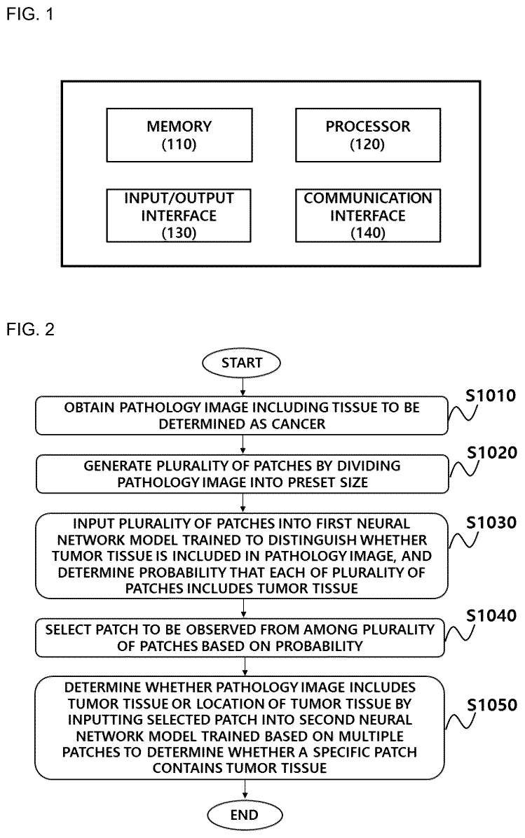

FIG. 2 is a flowchart of an operation performed by the apparatus 100, according to an embodiment. The operation of the apparatus 100 according to the embodiment of FIG. 2 can be understood as an operation performed by the processor 120.

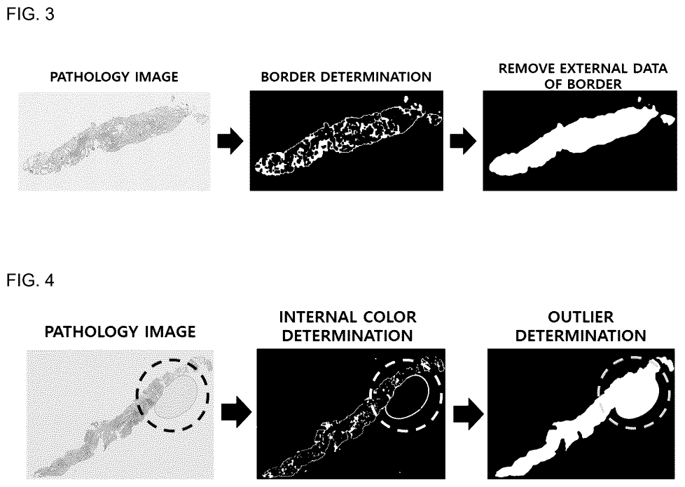

Each operation disclosed in FIG. 2 is only a preferred embodiment for achieving the purpose of the present invention, and some operations may be added or deleted as needed, and one operation may be included in another operation and performed. The order of each operation disclosed in FIG. 3 is only the order arranged for the convenience of understanding, but is not limited to a chronological order, and the order may be changed and operated according to the designer's choice.

Referring to FIG. 2, in operation S1010, the apparatus 100 may obtain a pathology image. For example, the apparatus 100 may obtain a pathology image from an external device or a linked device (e.g., a database, a photographing device, etc.). For example, the pathology image may include tissue that is a target for determining renal cancer, bladder cancer, or thyroid cancer, and the tissue may be dyed with a certain dye to be distinguished from other objects in the image.

In operation S1020, the apparatus 100 may generate a plurality of patches by dividing the pathology image into a preset size. Embodiments of generating a plurality of patches are as shown in FIGS. 3 to 7 below.

FIG. 3 is an exemplary view of an operation of removing data other than tissue by recognizing the border of tissue, according to an embodiment.

Referring to FIG. 3, in operation S1020, the apparatus 100 may determine a border of tissue included in the pathology image, remove data of an external area of the border of the tissue, and divide an internal area of the border of the tissue into a preset size to generate a patch. For example, the apparatus 100 may extract a border of a foreground, not a background, from the pathology image through a GrabCut algorithm, and may make the external area of the border null by removing data. Accordingly, the apparatus 100 may generate a patch so that only an area corresponding to the tissue is included in the patch.

When the pathology image is directly divided and a patch is generated without the process of FIG. 3, the external area of the tissue is also generated as a patch image, and unnecessary operations may be performed for training or utilizing a neural network. Therefore, the embodiment of the present invention may reduce resource consumption for utilizing the neural network by preprocessing the pathology image through the process of FIG. 3 so that only the data absolutely necessary for determining the presence or absence of cancer tissue may be used.

FIG. 4 is an exemplary view of an operation of filtering and removing border recognition of an outlier such as a bubble that occurred in tissue in the embodiment of FIG. 3.

Referring to FIG. 4, when recognizing a border, due to bubbles generated during a tissue examination, an area unnecessary for the examination may be recognized as a border. In this case, the apparatus 100 may additionally remove an area that does not include staining color information from an internal area of the border of the tissue. For example, the apparatus 100 may extract a border of a foreground, not a background, from the pathology image through a GrabCut algorithm, and may extract color information for the internal area of the border. At this time, the interior of an area corresponding to the tissue includes staining information of the tissue, but the interior of an area corresponding to the bubble does not include staining information of the tissue. Therefore, the apparatus 100 may extract color information for an internal area of a recognized border, recognize a border that does not include staining information as an outlier (e.g., bubble), and remove data of an area corresponding to the outlier.

When the pathology image is divided and a patch is generated without the process of FIG. 4, the area corresponding to the bubble may also be included in the patch image, which may cause unnecessary operations to be performed for training or utilization of a neural network. Therefore, the embodiment of the present invention may reduce resource consumption for utilizing the neural network by preprocessing the pathology image through the process of FIG. 4 so that only the data absolutely necessary for determining the neural network may be used.

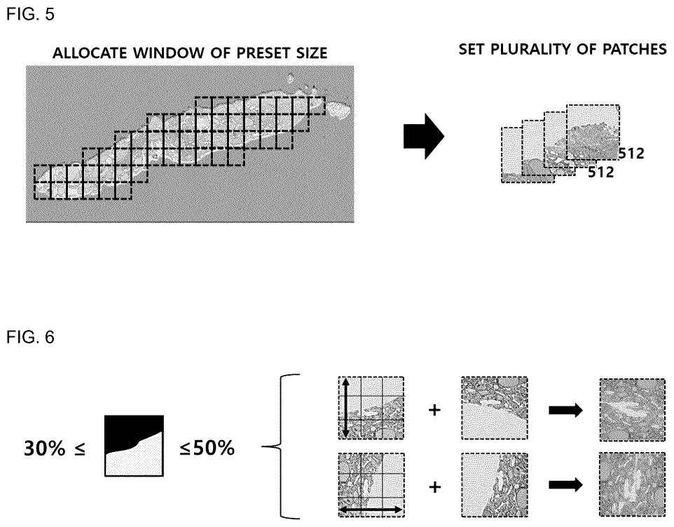

FIG. 5 is an exemplary view of an operation of dividing an area corresponding to tissue in a pathology image into a preset size and generating a plurality of patches, according to an embodiment.

Referring to FIG. 5, the apparatus 100 may allocate a window of 512×512 pixels (e.g., a red square on the left side of FIG. 5) to an area recognized as tissue in the pathology image, and may generate a patch (e.g., a black dotted square on the right side of FIG. 5) including an image inside each window. At this time, a patch captured from a window located in an internal area of the tissue from among arranged windows completely includes the tissue area, but a patch captured from a window located in a border of the tissue from among the windows may partially include an external area (e.g., a null area) of the tissue.

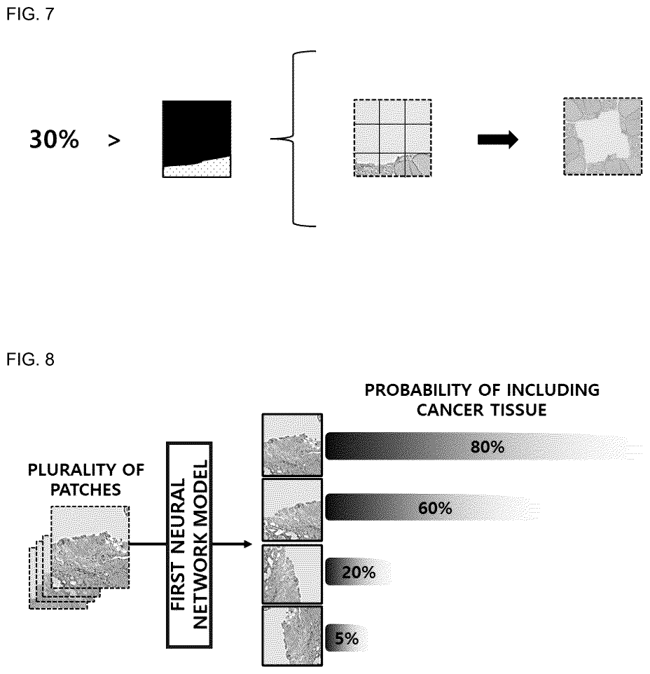

In this way, in the case of a patch including less than 50% of an internal area of tissue from among a plurality of patches, information about a tissue area that is an actual target of determination is small, so when the patch is utilized in a neural network, an error in the determination of a neural network may occur. For this reason, when a ratio of an internal area of tissue in a patch is less than a certain ratio, the following embodiment of FIG. 6 or FIG. 7 may be applied.

FIG. 6 is a view of an embodiment of modifying a patch when a tissue area included in a patch is 30% or more and 50% or less.

Referring to FIG. 6, in the case of a patch that includes 30% or more and 50% or less of an internal area of tissue from among patches generated by allocating a window, an area where the tissue area included in the patch is located may be symmetrical left and right or symmetrical up and down to enhance the tissue area included in the patch.

For example, the apparatus 100 may recognize an area where a tissue area is located by dividing a square of a patch into nine equal parts, and determine left and right symmetry or up and down symmetry in a direction where the tissue area increases to enhance the tissue area in the patch.

For example, in the case of an upper right of FIG. 6, when the square of the patch is divided into nine equal parts, because most of the tissue is located on a lower side of the patch, the tissue area in the patch may be enhanced by determining up and down symmetry.

For example, in the case of a lower right of FIG. 6, when the square of the patch is divided into nine equal parts, because most of the tissue is located on a left side of the patch, the tissue area in the patch may be enhanced by determining left and right symmetry.

FIG. 7 is a view of an embodiment of modifying a patch when a tissue area included in a patch is less than 30%.

Referring to FIG. 7, in the case of a patch that includes 30% or less of an internal area of tissue from among patches generated by allocating a window, the tissue area included in the patch may be copied and pasted into a blank area to enhance the tissue area in the patch.

For example, in the case of FIG. 7, when a square of a patch is divided into nine equal parts, a lower area where tissue is located may be copied to another divided area to enhance the tissue area in the patch.

In operation S1030, the apparatus 100 may input a plurality of patches preprocessed in operation S1020 into a first neural network model trained to distinguish whether tumor tissue is included in a pathology image, and may determine the probability that each of the plurality of patches includes tumor tissue.

The first neural network model of the present invention determines only whether tumor tissue is present in the pathology image, and proceeds to the next operation only when the probability that tumor tissue is included in the pathology image is high, and does not proceed to the next operation when the probability that tumor tissue is included in the pathology image is low, thereby reducing resource consumption of a neural network model.

FIG. 8 is an exemplary view of a first neural network model that determines the probability that a specific instance is included in a patch, according to an embodiment.

Referring to FIG. 8, the first neural network model may be composed of a neural network with a multiple instance learning (MIL) structure. An MIL neural network, when data to be determined is given, is a neural network that is strong in determining only whether a specific class is included in the data.

To this end, the first neural network model may be trained based on training data labeled with a single BAG class that only specifies whether a pathology image includes an instance corresponding to tumor tissue. For example, the first neural network model may be trained only whether a pathology image includes an instance corresponding to tumor tissue through training data labeled with only two BAG classes: “Class 1” for pathology images including tumor tissue, and “Class 0” for pathology images not including tumor tissue. The first neural network model that has completed training may output the probability that input data includes an instance corresponding to tumor tissue.

At this time, because the first neural network model of the present invention receives divided patches rather than the entire pathology image, the first neural network model may reduce resource consumption by omitting determination on blank images, and by distinguishing only patches with a high probability of including tumor tissue, more focused and precise observation is possible when utilizing the patches in the next second neural network model.

In operation S1040, the apparatus 100 may select a patch to be observed from among a plurality of patches based on the probability determined by the first neural network model for each patch. For example, only patches generated in operation S1020 with a high probability of including tumor tissue may be utilized in the next second neural network model, and if all patches generated in operation S1020 have a probability of including tumor tissue determined by the first neural network model below a preset probability, the apparatus 100 may skip utilizing the patches in the second neural network model and conclude that there is no tumor tissue in a corresponding pathology image.

On the other hand, if there is a patch from among the patches generated in operation S1020 that has a probability of including tumor tissue determined by the first neural network model that is greater than a preset probability, in operation S1050, the apparatus 100 may select the patch that has a probability of including tumor tissue that is greater than a preset probability and input the patch into the second neural network model.

This is similar to when a specialist observes a pathology image, if there is a part suspected of being tumor tissue, he or she observes that part with another specialist to make a more accurate conclusion, or conversely, if there is no part suspected of being tumor tissue from the beginning, the specialist quickly concludes that there is no probability of cancer without further discussion with another specialist.

At this time, the second neural network model is designed and trained in a different way from the first neural network model, so that the second neural network model may determine whether a patch includes tumor tissue using a different determination method from that of the first neural network model.

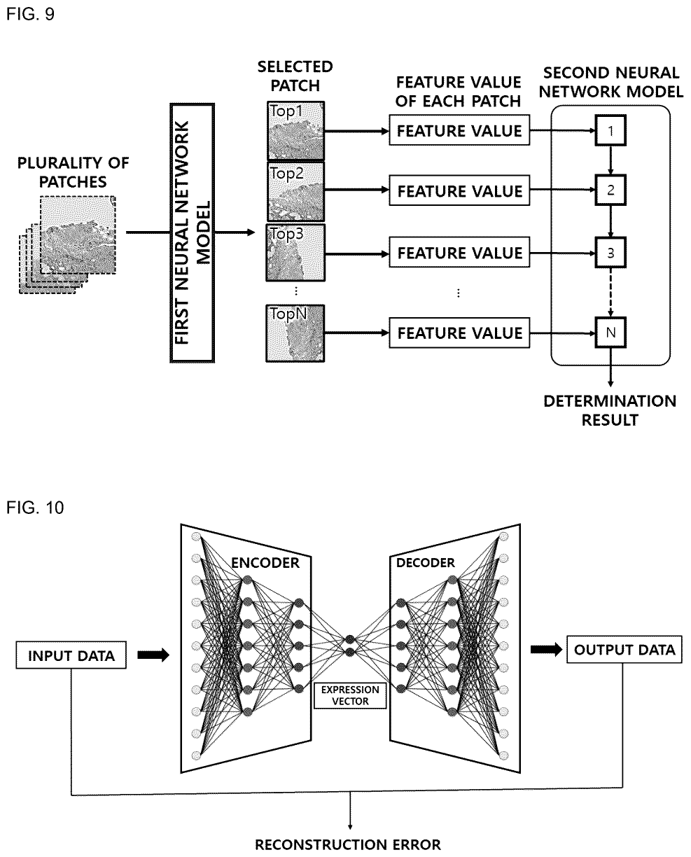

An embodiment of the present invention presents the form of a second neural network model capable of operating in conjunction with the first neural network model through the following FIGS. 9 to 13.

FIG. 9 is an exemplary view of a second neural network model configured in the form of a recurrent neural network (RNN) and operating, according to an embodiment.

Referring to FIG. 9, the second neural network model may be composed of a neural network with an RNN structure. When data with a time-series meaning in general is given, the RNN has the advantage of determining data by considering changes according to time-series order. In addition, the RNN may determine whether tumor tissue is included by identifying not only the time-series order but also a spatial relationship and order changes of input patches. For example, in the case of training the second neural network model, by inputting a plurality of patches generated by dividing a single pathology image including tumor tissue into an RNN in the order of patches including more tumor tissue, and training the second neural network model whether a patch includes tumor tissue, the second neural network model may be trained whether a patch includes tumor tissue through a spatial relationship and order changes of the patches. At this time, when training the RNN, an input value input to an input layer of the RNN may utilize a feature value extracted from an MIL neural network for a specific patch, and an output value input to an output layer of the RNN may be set to be a class of whether cancer tissue is included (included: 1, not included: 0).

Accordingly, the apparatus 100 may arrange patches in which probabilities determined in operation S1040 are greater than a certain threshold in order of high probability, and sequentially input a feature value extracted from the first neural network model for a corresponding patch to the second neural network model, thereby outputting the probability that a corresponding pathology image includes tumor tissue.

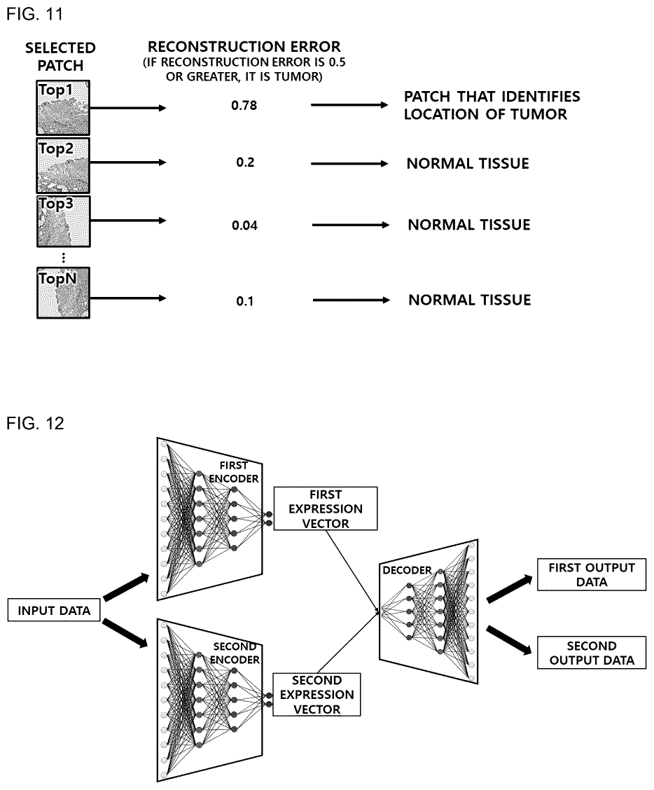

FIGS. 10 and 11 are exemplary views of a second neural network model configured in the form of an autoencoder including one encoder and one decoder and operating, according to an embodiment.

Referring to FIG. 10, the second neural network model may be composed of a neural network with an autoencoder structure including one encoder and one decoder. In FIG. 10, when certain input data is input, the encoder compresses the input data as much as possible (=expression vector), and the decoder restores the compressed data back to the original input data form based on the features of the compressed data. The autoencoder of the second neural network model according to the embodiment of FIG.

10 may be trained to encode and decode input data based on training data of a pathology image including only normal tissue to restore the input data. In this case, the autoencoder of the second neural network model according to the embodiment of FIG. 10 is trained to perform restoration well when a pathology image including normal tissue is input, but to have poor restoration capability when a pathology image including abnormal tissue (e.g., tumor tissue) is input. In the autoencoder, a difference between original input data and restored output data is called a reconstruction error.

Referring to FIG. 11, the autoencoder of the second neural network model according to the embodiment of FIG. 10 will have poor restoration capability for a pathology image including abnormal tissue, so when the patch selected in operation S1040 is input and encoding and decoding are performed, the apparatus 100 may determine a patch having a restoration error greater than a preset value (e.g., 0.5 in FIG. 11) by a location of tumor tissue in a pathology image.

FIGS. 12 to 14 are exemplary views of a second neural network model configured in the form of an autoencoder including two encoders and one decoder and operating, according to an embodiment.

Referring to FIG. 12, the second neural network model may be composed of a neural network with an autoencoder structure including two encoders and one decoder. In FIG. 12, when certain input data is input, the first encoder and the second encoder compress input data, and the decoder restores the compressed data back to the original input data form based on the features of the compressed data.

In the autoencoder of the second neural network model according to the embodiment of FIG. 12, the first encoder and the second encoder may be trained based on different training data or may have different layer structures.

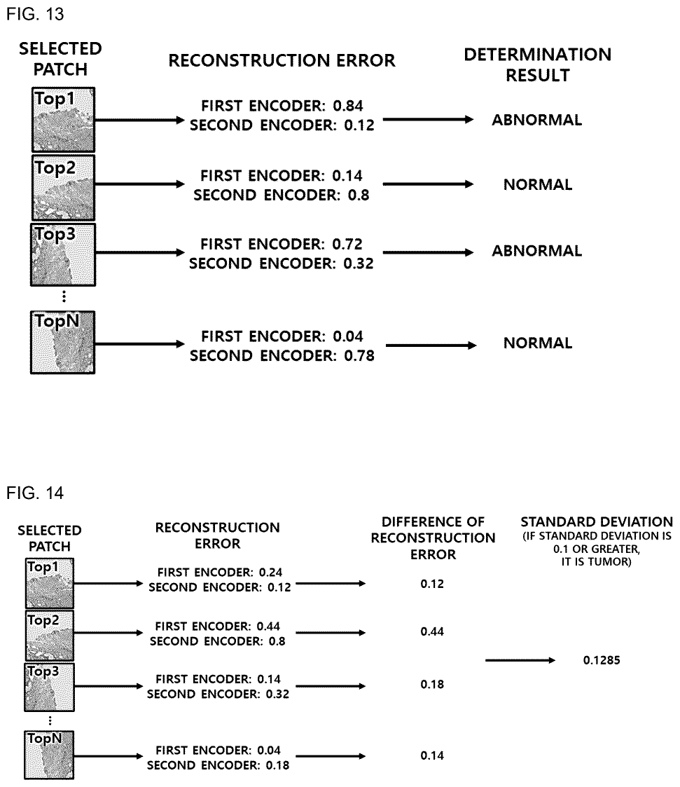

FIGS. 13 and 14 show embodiments where the first encoder and the second encoder are trained differently.

First, according to the embodiment of FIG. 13, in a neural network structure of the autoencoder according to the embodiment of FIG. 12, the first encoder trains a parameter to encode input data based on training data of a pathology image including only normal tissue, and the second encoder trains a parameter to encode input data based on training data of a pathology image including only abnormal tissue. Accordingly, for identical input data, a first expression vector generated by the first encoder and a second expression vector generated by the second encoder may have different feature values.

That is, in the embodiment of FIG. 13, linkage of the first encoder and the decoder is trained to perform restoration well for a patch including only normal tissue, but not to perform restoration properly for a patch including abnormal tissue. Linkage of the second encoder and the decoder is trained to perform restoration well for a patch including abnormal tissue, but not to perform restoration properly for a patch including only normal tissue. Accordingly, depending on whether an image including only normal tissue or an image including abnormal tissue is input, the first encoder and the second encoder exhibit different performances, and results of those performances may be confirmed through a restoration error between the input data and first output data and a restoration error between the input data and second output data.

Therefore, in the embodiment of FIG. 13, when a patch is input, if a first restoration error through the linkage of the first encoder and the decoder is greater than a second restoration error through linkage of the second encoder and the decoder, the apparatus 100 may determine that the patch is abnormal (=including tumor tissue) and determine a location corresponding to the patch as a location of a tumor. In addition, when a patch is input, if the first restoration error through the linkage of the first encoder and the decoder is less than the second restoration error through the linkage of the second encoder and the decoder, the apparatus 100 may determine that the patch is normal (=excluding tumor tissue).

Next, according to the embodiment of FIG. 14, in the neural network structure of the autoencoder according to the embodiment of FIG. 12, both the first and second encoders may train their respective parameters to encode input data based on training data of a pathology image including only normal tissue. At this time, the first encoder and the second encoder may be trained through training data with samples of different normal tissues, or may be designed to have different layer structures. Accordingly, for identical input data, the first expression vector generated by the first encoder and the second expression vector generated by the second encoder may have different feature values.

In this case, in the embodiment of FIG. 14, both the first encoder and the second encoder are trained to perform restoration well when a pathology image including normal tissue is input, but to have poor restoration capability when a pathology image including abnormal tissue (e.g., tumor tissue) is input. Accordingly, because the first encoder and the second encoder according to the embodiment of FIG. 14 have the ability to compress and restore a pathology image including normal tissue, the standard deviation of respective restoration errors is not large. In contrast, because the first encoder and the second encoder are not trained to compress and restore a pathology image including abnormal tissue (e.g., tumor tissue), the standard deviation of respective restoration errors is large.

Therefore, referring to FIG. 14, if the standard deviation for difference values of a restoration error of the first expression vector and a restoration error of the second expression vector respectively generated by the two encoders is greater than or equal to a preset value (e.g., 0.1 in FIG. 13).

According to the embodiments described above, the present invention may more effectively handle the diversity and complexity of cancer tissues by linking two or more neural networks trained in different ways and using them for cancer diagnosis, and may alleviate an overfitting problem of a neural network model and improve the generalization ability.

In addition, because the neural network model of the present invention may be trained in different ways for an identical training data set, the computation and resources required for training and executing the model may be reduced, thereby improving practicality, and because neural network models with different special features are linked, a diagnosis may be made by considering various characteristics of cancer tissues.

Therefore, the present invention may greatly contribute to the development of medical technology by achieving practical application of deep learning and machine learning technology in the field of histopathological examination and at the same time greatly improving the accuracy and efficiency of cancer tissue diagnosis.

The embodiments described above may be implemented by hardware components, software components, and/or any combination thereof. For example, the devices, the methods, and components described in the embodiments may be implemented by using general-purpose computers or special-purpose computers, such as a processor, a controller, an arithmetic logic unit (ALU), a digital signal processor, a microcomputer, a field programmable gate array (FPGA), a programmable logic unit (PLU), a microprocessor, or any other devices which may execute and respond to instructions. A processing apparatus may execute an operating system (OS) and a software application executed in the OS. Also, the processing apparatus may access, store, operate, process, and generate data in response to the execution of software. For convenience of understanding, it may be described that one processing apparatus is used. However, one of ordinary skill in the art will understand that the processing apparatus may include a plurality of processing elements and/or various types of processing elements. For example, the processing apparatus may include a plurality of processors or a processor and a controller. Also, other processing configurations, such as a parallel processor, are also possible.

The software may include computer programs, code, instructions, or any combination thereof, and may construct the processing apparatus for desired operations or may independently or collectively command the processing apparatus. In order to be interpreted by the processing apparatus or to provide commands or data to the processing apparatus, the software and/or data may be permanently or temporarily embodied in any types of machines, components, physical devices, virtual equipment, computer storage mediums, or transmitted signal waves. The software may be distributed over network coupled computer systems so that it may be stored and executed in a distributed fashion. The software and/or data may be recorded in a computer-readable recording medium.

A method according to an embodiment may be implemented as program instructions that can be executed by various computer devices, and recorded on a computer-readable recording medium. The computer-readable recording medium may include program instructions, data files, data structures or a combination thereof. Program instructions recorded on the medium may be particularly designed and structured for embodiments or available to one of ordinary skill in a field of computer software. Examples of the computer-readable recording medium include magnetic media, such as a hard disc, a floppy disc, and magnetic tape; optical media, such as a compact disc-read only memory (CD-ROM) and a digital versatile disc (DVD); magneto-optical media, such as floptical discs; and hardware devices specially configured to store and execute program instructions, such as ROM, random-access memory (RAM), a flash memory, etc. Program instructions may include, for example, high-level language code that can be executed by a computer using an interpreter, as well as machine language code made by a complier.

In concluding the detailed description, those skilled in the art will appreciate that many variations and modifications may be made to the preferred embodiments without substantially departing from the principles of the present invention. Therefore, the disclosed preferred embodiments of the invention are used in a generic and descriptive sense only and not for purposes of limitation.

1. A method of performing a cancer metastasis tissue determination apparatus operated by a processor, the method comprising:
obtaining a pathology image including tissue to be determined as cancer;
generating a plurality of patches by dividing the pathology image into a preset size;
determining a probability that each of the plurality of patches includes tumor tissue by inputting the plurality of patches into a first neural network model trained to distinguish whether the pathology image includes tumor tissue;
selecting a patch to be observed from among the plurality of patches based on the probability; and
determining whether the pathology image includes tumor tissue or a location of the tumor tissue by inputting the selected patch into a second neural network model trained based on multiple patches to determine whether a specific patch contains tumor tissue.

2. The method of claim 1, wherein the first neural network model is composed of a neural network with a multiple instance learning (MIL) structure and trained based on training data labeled with a single BAG class that only specifies whether the pathology image includes an instance corresponding to tumor tissue, and outputs a probability that input data includes the instance.

3. The method of claim 2, wherein the selecting a patch to be observed comprises:
classifying patches determined to have the probability greater than a certain threshold; and
arranging the classified patches in order of high probability.

4. The method of claim 3, wherein the second neural network model is composed of a neural network with a recurrent neural network (RNN) structure and trained to determine whether tumor tissue is included in a patch by identifying changes in order of input patches and a spatial relationship between the input patches, and outputs a probability that the classified patches include tumor tissue when the classified patches are input in order in which they are arranged.

5. The method of claim 2, wherein the second neural network model is composed of a neural network with an autoencoder structure including an encoder and a decoder and trained to encode and decode input data based on training data of a pathology image including only normal tissue and restore the input data, and determines a patch in which a restoration error is greater than a preset value when receiving the selected patch and performing encoding and decoding by a location of tumor tissue in the pathology image.

6. The method of claim 2, wherein the second neural network model is composed of a neural network with an autoencoder structure including two encoders and one decoder trained based on different training data and trained to encode and decode input data based on training data of a pathology image including only normal tissue and restore the input data, and determines that the pathology image includes tumor tissue if standard deviation for difference values of respective restoration errors by the two encoders is greater than or equal to a preset value when receiving the selected patch and performing encoding and decoding.

7. The method of claim 1, wherein the generating a plurality of patches comprises:
determining a border of tissue included in the pathology image;
removing data of an external area of the border of the tissue; and
generating a patch by dividing an internal area of the border of the tissue into a preset size.

8. The method of claim 7, wherein the generating a plurality of patches, after generating the patch, comprises:
when a tissue area included in the patch is 30% or more and 50% or less of the patch, making the tissue area included in the patch symmetrical left-right or up-down within the patch.

9. The method of claim 7, wherein the generating a plurality of patches, after generating the patch, comprises:
when a tissue area included in the patch is less than 30% of the patch, copying the tissue area included in the patch and pasting the tissue area into a blank area.

10. A cancer metastasis tissue determination apparatus comprising:
a memory including an instruction; and
a processor for performing a certain operation based on the instruction,
wherein the operation of the processor comprises:
obtaining a pathology image including tissue to be determined as cancer;
generating a plurality of patches by dividing the pathology image into a preset size;
determining a probability that each of the plurality of patches includes tumor tissue by inputting the plurality of patches into a first neural network model trained to distinguish whether the pathology image includes tumor tissue;
selecting a patch to be observed from among the plurality of patches based on the probability; and
determining whether the pathology image includes tumor tissue or a location of the tumor tissue by inputting the selected patch into a second neural network model trained to determine whether the plurality of patches include tumor tissue.
